(19) United States
(12) Patent Application Publication
(10) Pub. No.: US 2023/0138721 A1
(43) Pub. Date: May 4, 2023

(54) MESOPOROUS SILICA NANOPARTICLES, METHODS OF MAKING, AND METHODS OF USING

(71) Applicant: UNM RAINFOREST INNOVATIONS, Albuquerque, NM (US)

(72) Inventors: Rita E. Serda, Albuquerque, NM (US); Jimin Guo, Albuquerque, NM (US); Achraf Noureddine, Albuquerque, NM (US); Stefan A. Franco, Albuquerque, NM (US); Henning DeMay, Albuquerque, NM (US)

(21) Appl. No.: 17/916,115

(22) PCT Filed: Mar. 30, 2021
(86) PCT No.: PCT/US2021/024788

§ 371 Date: Sep. 30, 2022

Related U.S. Application Data
(60) Provisional application No. 63/001,893, filed on Mar. 30, 2020.

(51) Int. Cl. A61K 47/69; C01B 33/18; A61K 47/64; A61K 47/61; A61K 47/60

(52) U.S. Cl. CPC A61K 47/6929; C01B 33/18; A61K 47/645; A61K 47/61; A61K 47/60; C01P 2004/64; C01P 2004/04

(57) ABSTRACT

A mesoporous silica nanoparticle includes a plurality of pores, a bioactive agent disposed in at least a portion of the ores, and an immunomodulatory moiety either disposed in a portion of the pores or bound to at least a portion of the outer surface of the nanoparticle.

### CROSS-REFERENCE TO RELATED APPLICATION

This application claims the benefit of U.S. Provisional Patent Application No. 63/001,893, filed Mar. 30, 2020, which is incorporated herein by reference in its entirety.

### SUMMARY

This disclosure describes, in one aspect, a mesoporous silica nanoparticle. Generally, the mesoporous silica nanoparticle includes a plurality of pores, a bioactive agent disposed in at least a portion of the pores, and an immunomodulatory moiety either disposed in a portion of the pores or bound to at least a portion of the outer surface of the nanoparticle.

In some embodiments, the mesoporous silica nanoparticle includes a cationic layer disposed on at least a part of the outer surface and the immunomodulatory moiety is bound to at least a portion of the cationic layer.

In some embodiments, the mesoporous silica nanoparticle includes a cationic layer disposed on at least a part of the outer surface, and an anionic layer disposed on at least a portion of the cationic layer, and the immunomodulatory moiety is bound to at least a portion of the anionic layer.

In some embodiments, the immunomodulatory moiety includes a pathogen-associated molecular pattern (PAMP), a danger-associated molecular molecule (DAMP), a cytokine, an antibody, another immunogenic entity, or a combination thereof. In some of these embodiments, the PAMP can include lipopolysaccharide (LPS), monophosphoryl lipid A (MPL), CpG, R-848, or PolyIC.

In embodiments, having a cationic layer, the cationic layer can include polyethyleneimine (PEI), chitosan, poly(L-lysine), poly(γ-glutamic acid), or a cationic lipid.

In embodiments having an anionic layer, the anionic layer can include alginate, poly(D-lactic acid), poly(acrylic acid), dextran sulphate, or hyaluronic acid.

In another aspect, this disclosure describes a pharmaceutical composition that includes any embodiment of the mesoporous silica nanoparticle summarized above.

In another aspect, this disclosure describes a method for treating a subject having a tumor. Generally, the method includes administering to the subject a composition that includes any embodiment of the mesoporous silica nanoparticle summarized above in an amount effective to ameliorate at least one symptom or clinical sign of having the tumor.

In some embodiments, the mesoporous silica nanoparticle is administered intratumorally. In other embodiments, the mesoporous silica nanoparticle is administered intraperitoneally.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Activation of TLRs by TLR ligand silicified cancer cells. TLR ligands on the surface of particulates (e.g. cells, nanoparticles) engage receptors both the surface and within immune cells activating diverse signaling pathways.

FIG. 2. Transmission electron microscopy (TEM) image of a mesoporous silica nanoparticle (MSN). Scale bar=0.2 μm. (B)

FIG. 3. Quantity and stability of fluorescent ligand binding to mesoporous silica nanoparticles with consecutive washes.

FIG. 4. Immunogenic mesoporous silica nanoparticles (MSN). Mass formulation of MSNs with (left) or without (right) Si content.

FIG. 5. Surface potential of MSN with variable surface coating.

FIG. 6. Immunogenic mesoporous silica nanoparticles (MSN) kill cancer cells. BR5-Akt or ID8 ovarian cells were incubated with silicified cancer cells in the presence of YOYO-3 and either silicified cancer cells or MSN (both coated with TLR ligands). YOYO-3 is an indicator of cell death and it is absorbed by silica. As seen at time 0 in the merged bright field and fluorescent images of BR5 cells, both the silicified cell vaccine and the MSN were positive for YOYO-3. The larger size of the silicified cancer cells gave them a strong fluorescent signal which was stable over time for both BR5-Akt and ID8 cells (graphs to the right, blue lines), indicating a lack of cell death induced directly by the vaccine. In addition, bright field images support proliferation of cancer cells in the presence of the silicified cancer cells. In contrast, MSN caused death of the majority of the cancer cells within 15-20 hours (as seen by uptake of YOYO-3 in the dead cells and by the graphs (red lines).

FIG. 7. Cancer cell proliferation (left) and bone marrow-derived dendritic cell (BMDC, right) proliferation in the presence of increasing amounts of mesoporous silica nanoparticles (MSNs). Relative BR5-Akt ovarian cancer cell (left) or BMDC (right) proliferation in the presence of increasing concentrations of control or TLR ligand-bound MSN at 24 hours.

FIG. 8. Biodistribution of TLR ligand-MSN in mice with ovarian cancer. Fluorescence images of excised liver (LV), kidney (KD), lung (LU), heart (HT), spleen (SP), mesenteric lymph node (LN-M), subcutaneous lymph nodes (LN-SC), and omentum (OM) 24 hours after intraperitoneal administration of MSN-PEI-CpG-MPL.

FIG. 9. Biodistribution of TLR ligand-MSN in mice with ovarian cancer. Fluorescent intensity per tissue (left) and fluorescence intensity per gram of tissue (right).

FIG. 10. Biodistribution of TLR ligand-MSN in mice with ovarian cancer. Relative fluorescence signal per organ.

FIG. 11. In vivo association of bone marrow-derived dendritic cells with mesoporous silica nanoparticles (MSNs) displaying TLR ligands. Percentage of CD11⁺ dendritic cells associated with MSN from the entire cell population of cells harvested from peritoneal fluid, mesenteric lymph node (M lymph), subcutaneous lymph nodes (SC lymph), and spleen 24 hours after intraperitoneal injection of MSN-PEI-CpG-MPL in female FVB mice (n=3/group).

FIG. 12. MSN-driven elimination of established tumors in mice with ovarian cancer. Female FVB mice were administered BR5-Akt-Luc2 ovarian cancer cells (false-colored based on bioluminescence) by intraperitoneal (IP) injection. When tumors were established, MSN were introduced into the tumor microenvironment (IP) or subcutaneously (SC). Within 15-20 days, most of the tumors were eliminated following IP injection (MSN-PEI-MPL-CPG/IP). In contrast, SC injection of MSNs did not eliminate tumors (MSN-PEI-MPL-CPG/SC). Injection of MSN without TLR ligands did not alter tumor growth (MSN IP).

FIG. 13. TLR ligand-MSN driven elimination of existing tumors in mice with ovarian cancer. Tumor burden based on cancer cell bioluminescence in female FVB mice bearing pre-existing BR5-Akt-Luc2 ovarian tumors following intraperitoneal administration of control or TLR ligand-bound MSN.

FIG. 14. TLR ligand-MSN driven elimination of existing tumors in mice with ovarian cancer. Tumor burden and survival in female FVB mice bearing pre-existing BR5-Akt-Luc2 ovarian tumors following intraperitoneal administration of control or TLR ligand-bound MSN.

### DETAILED DESCRIPTION OF ILLUSTRATIVE EMBODIMENTS

This disclosure describes mesoporous silica nanoparticles modified to display immunomodulatory moieties on their surfaces or within their porous structure. The mesoporous silica nanoparticles may be formulated into a pharmaceutical composition. Administering the mesoporous silica nanoparticles to a subject that has cancer can elicit an enhanced immune response from the subject against cells of the cancer.

Pathogen-associated molecular patterns (PAMPs), such as monophosphoryl lipid A (MPL), derived from the bacterial endotoxin lipopolysaccharide are commonly used in vaccine formulations. These molecules activate Toll-like receptors (TLRs) on the surface of antigen presenting cells (APCs, e.g., dendritic cells (DC), macrophages), activating signaling pathways that upregulate expression of costimulatory molecules (e.g., CD40, CD80) and major histocompatibility complex (MEW), facilitating the ability of the APC to engage in effective immune responses. Attaching a TLR ligand to a solid surface creates a microbial mimic, able to stimulate internalization by APC and cell activation.

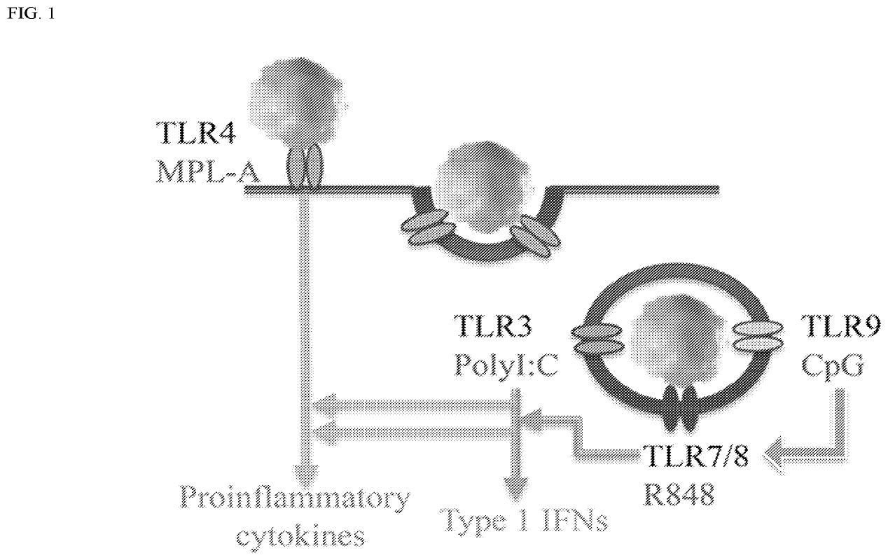

After a microbial mimic is internalized, PAMPs on the surface of the microbial mimic further activate TLRs (e.g., TLR-9, the receptor for CpG oligodeoxynucleotides) located within endosomes. Thus, attaching TLR ligands to the surface of particulates enables interactions with TLRs found inside of vesicles within immune cells, receptors that are not available to free ligand. In addition, cell/particulate bound TLR ligands cause multivalent activation of receptors, leading to greater activation of APCs. FIG. 1 shows internalization of a silicified cancer cell coated with TLR ligand and its ability to activate TLRs on both the surface and inside of immune cells.

One benefit of using cancer cells as microbial mimics is that the silicified cancer cell delivers patient-specific cancer antigens along with the TLR agonist, which drives an immune response specific for the silicified cancer cell by activating T cells that then kill cancer cells. Restricting the use of immunological adjuvants to the TLR agonist on the silicified cell/particulate surface also localizes immune activation, reducing systemic effects.

This disclosure describes mesoporous silica nanoparticles with secondary surface modification that allow presentation of immunomodulatory moieties. Surface modification of the mesoporous silica nanoparticles enable functionalization of the nanoparticle surface. Mesoporous silica nanoparticles can be coated to enhance decoration with pathogen-associated molecular pattern (PAMP), danger-associated molecular molecule (DAMP), or other immunomodulatory moieties. Silica-based surface modification enables surface binding of adjuvants or other immunomodulatory compounds. The native chemistry of the silica nanoparticles includes hydroxyl (silanol ≡Si—OH) groups. At physiological pH, the silanol groups are largely dehydroxylated creating an anionic (≡Si—O⁻) surface that adsorbs cationic molecules and polymers that, in turn, can adsorb and retain anionic ligands.

While described herein in the context of an exemplary embodiment in which the secondary surface modification includes polyethylenimine (PEI), the surface-modified mesoporous silica nanoparticles described herein can include a secondary surface modification using any suitable cationic, anionic, or hydrophobic modification. Exemplary suitable cationic surface modifications includes, but are not limited to polyethyleneimine (PEI), chitosan, poly(L-lysine), poly(γ-glutamic acid), cationic lipid, or other cationic molecule that increases binding of certain immunomodulatory moieties (e.g., PAMPs, CpG, MPL, LPS) to the surface of the mesoporous silica nanoparticle. Alternatively, if the immunomodulatory moiety that one wishes to bind to the surface of the mesoporous silica nanoparticle is cationic, one can use an anionic molecule for secondary modification. Exemplary anionic surface modifications include, but are not limited to, poly(D-lactic acid), poly(acrylic acid), dextran sulphate, or hyaluronic acid. Embodiments displaying one or more cationic immunomodulatory moieties can includes a first, cationic polymer layer, as described above, and an anionic polymer layer disposed on at least a portion of the cationic polymer layer. The cationic immunomodulatory moiety can then be adsorbed to the anionic polymer layer. Embodiments displaying one or more hydrophobic immunomodulatory moieties can includes a first, cationic polymer layer, as described above, and a hydrophobic polymer layer disposed on at least a portion of the cationic polymer layer. The hydrophobic immunomodulatory moiety can then be adsorbed to the hydrophobic polymer layer.

The coating provided by the polymer or molecule—whether cationic, anionic, or hydrophobic—need not be continuous or uniform. That is, the polymer may provide a discontinuous and/or uneven coating. In certain embodiments, however, the polymer can form a uniform layer that coats the entire mesoporous silica nanoparticle. When more than one layer is present, each layer can be continuous or discontinuous, independent of the character of any other layer.

Mesoporous silica nanoparticles that employ a cationic secondary surface modification and adsorbed PAMPs (e.g., TLR ligands) possesses multifaceted immunological properties. For example, they are able to both activate immune cells and directly kill cancer cells. When delivered into the tumor microenvironment, the PAMP-coated mesoporous silica nanoparticles lead to release of endogenous tumor antigens and activation of immune cells, stimulating effective anti-cancer immune responses. Thus, in clinical situations where autologous (patient) cancer cells are not available, TLR ligands can be bound to the surface (or on located within the pores, or located on the surface and within the pores) of nanoparticles (or microparticles).

While described herein in the context of exemplary embodiments in which the mesoporous silica nanoparticle is modified to be decorated with a TLR agonist (e.g., PEI, CpG, MPL, or LPS), the mesoporous silica nanoparticles, and methods of using mesoporous silica nanoparticles, described herein can involve mesoporous silica nanoparticles functionalized with one or more PAMPs, one or more DAMPs, or one or more alternative immunomodulatory moieties, or any combination of two or more PAMPs, DAMPS, and/or immunomodulatory moieties, as desired. Exemplary PAMPs include, but are not limited to, lipopolysaccharide (LPS), monophosphoryl lipid A (MPL), PolyIC, an imidazoquinoline amine (e.g., R-848), double-stranded RNA, lipoteichoic acid, peptidoglycan, a virus, and unmethylated CpG. DAMPS are endogenous molecules created upon tissue injury. Exemplary DAMPs include, but are not limited to, heat shock proteins, high mobility group box 1, proteins such as hyaluronan fragments, and non-protein targets such as ATP, uric acid, DNA and heparin sulfate.

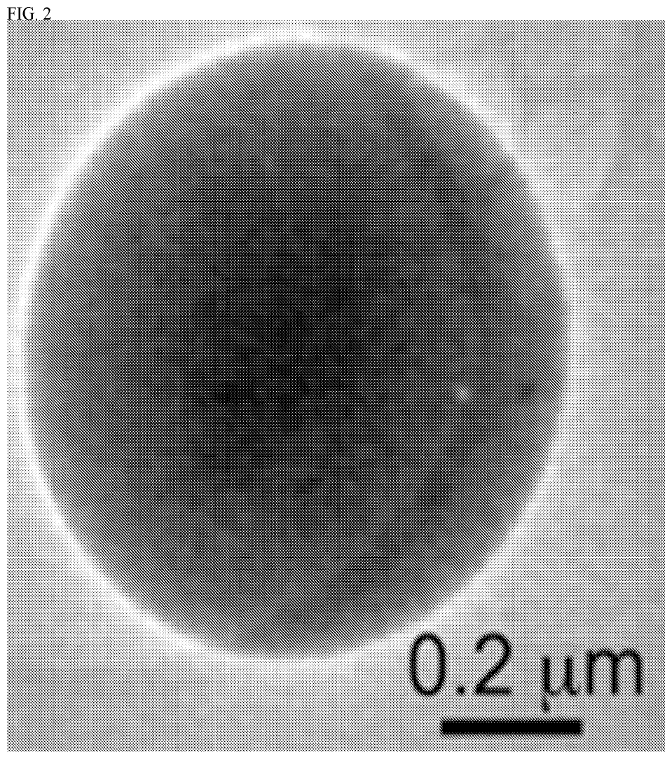

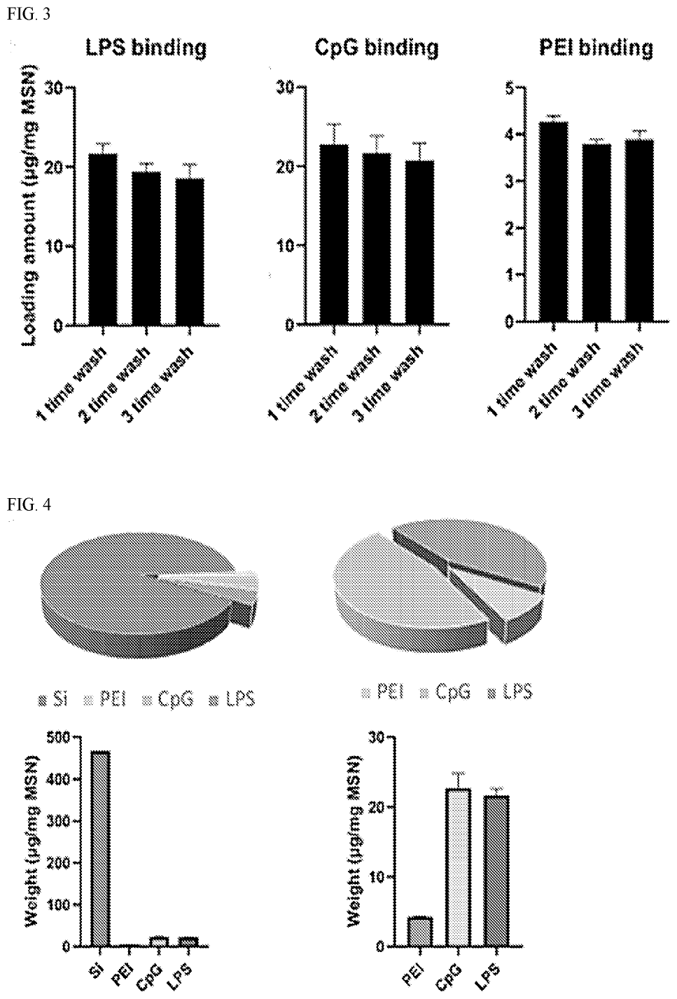

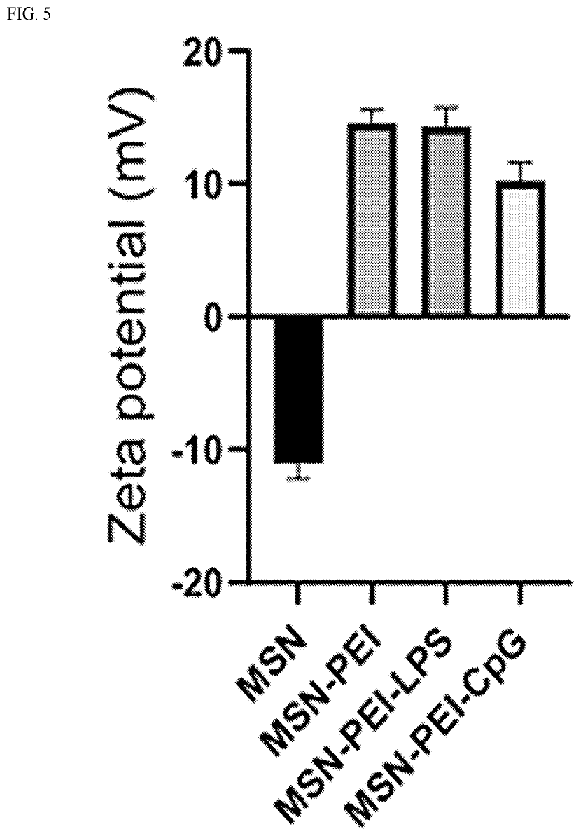

FIGS. 2-5 show a mesoporous silica nanoparticle (MSN, FIG. 2) and its characterization following surface coating with polyethylenimine (PEI) or PEI and TLR ligands (CPG, LPS). The ligand-coated particles are able to activate immune cells, helping to reverse immune suppression in the tumor microenvironment. In addition, based on the properties of the nanoparticles (e.g., size, charge), it is also possible to target and directly kill cancer cells.

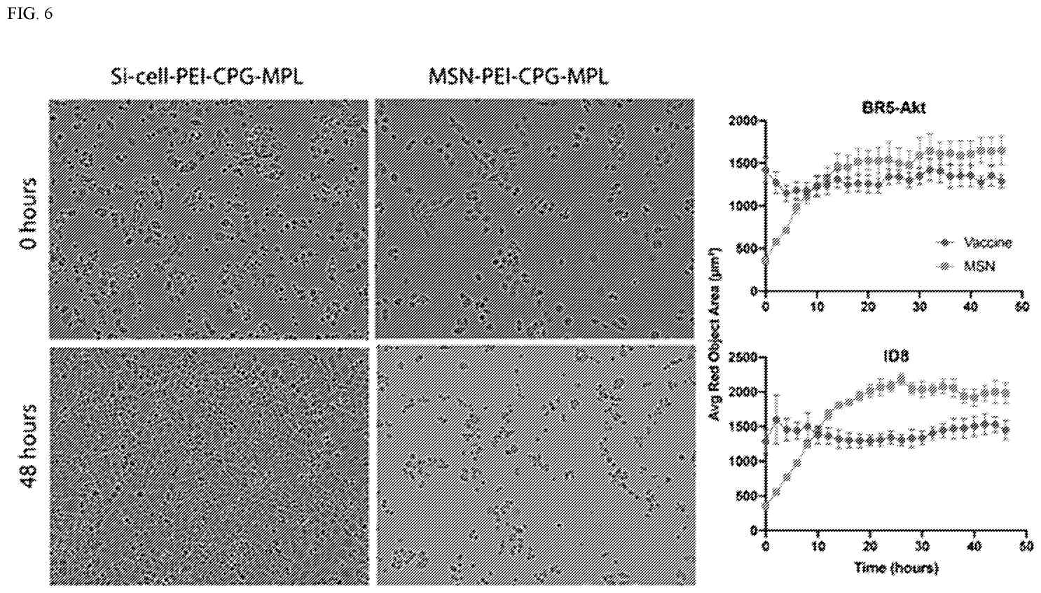

FIG. 6 shows that mesoporous silica nanoparticles coated with PEI, CPG, and MPL are internalized by and directly kill ovarian cancer cells, releasing cancer antigens that are then available to drive immune responses. In contrast, a silicified cancer cell vaccine works specifically on immune cells, delivering both adjuvant and antigen to stimulate anti-cancer immunity. The larger silicified cancer cells, as shown in FIG. 6, are not internalized by cancer cells and do not directly affect proliferation of the cancer cells.

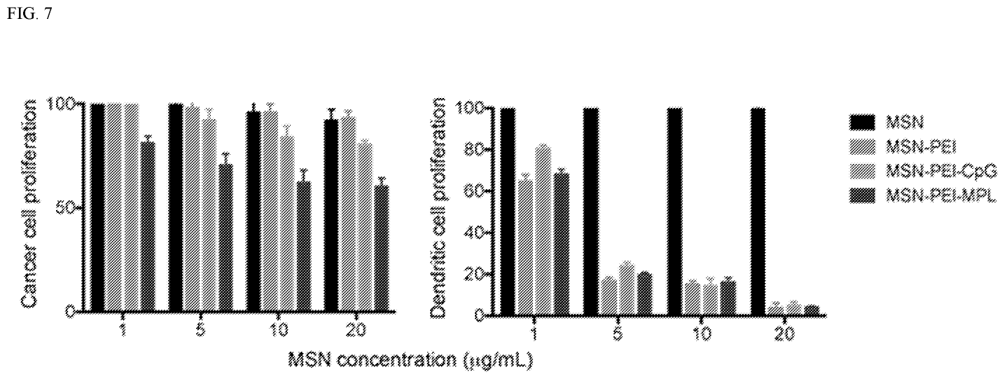

FIG. 7 is data from a proliferation assay that shows that while MSN-PEI-MPL (cationic nanoparticles containing MPL) display concentration dependent toxicity towards (BR5-Akt) cancer cells, all mesoporous silica nanoparticles with PEI in their formulation are highly toxic to dendritic cells. Thus, while direct cancer cells killing may be involved in the therapeutic effect of MSN particles, the main mechanism of action appears to be on immune cells.

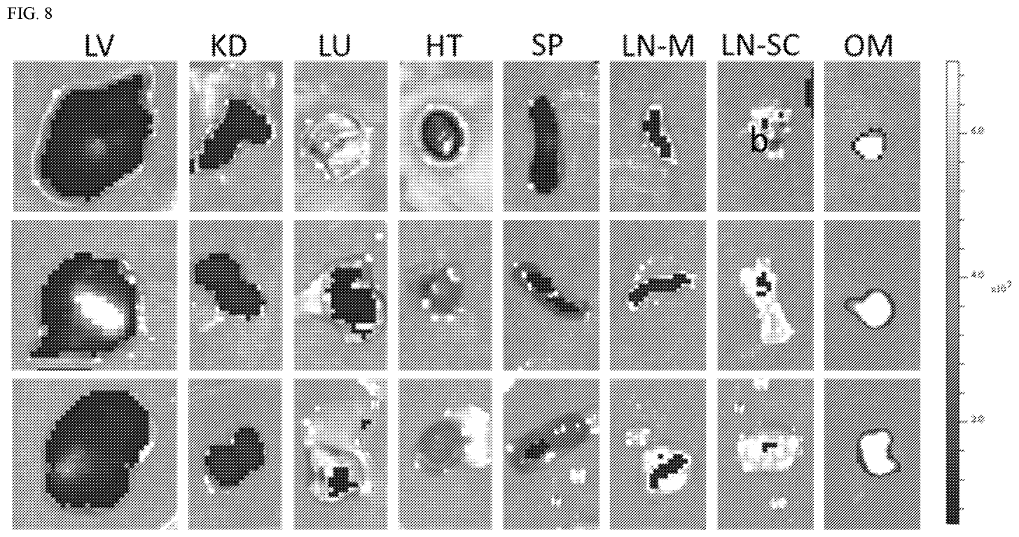

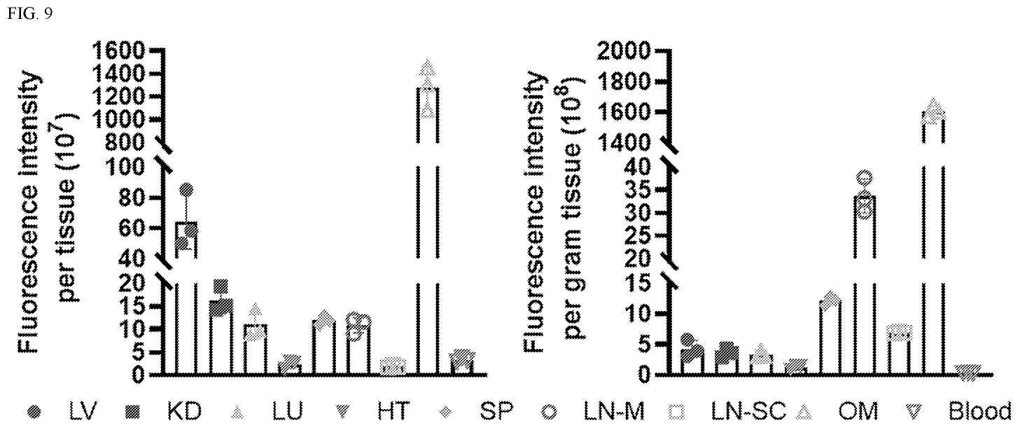

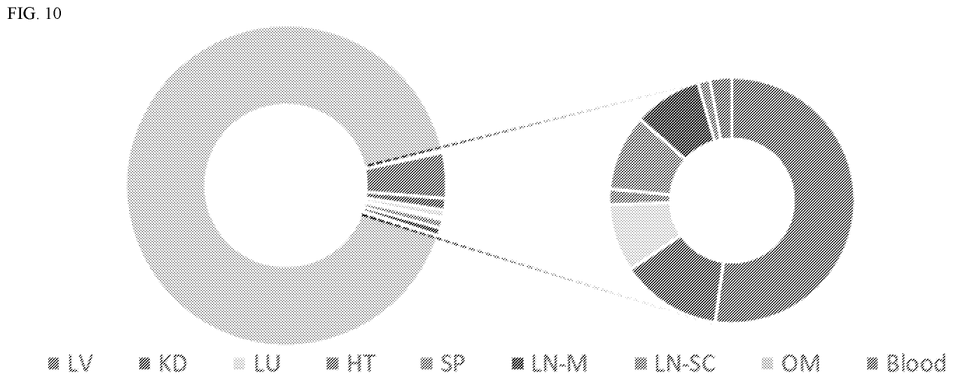

FIGS. 8-10 show that 24 hours following intraperitoneal injection in mice, 90% of MSN-PEI-CpG-MPL are located within the omentum. Filtering organs (liver, kidney) and lymphatic tissues (spleen and lymph nodes) also contain detectable amounts of MSN-PEI-CpG-MPL. The omentum is considered a protector of the peritoneal cavity with immune cells enriched within milky bodies, thus positioning MSN-PEI-CpG-MPL in tissues that support initiation of immune responses.

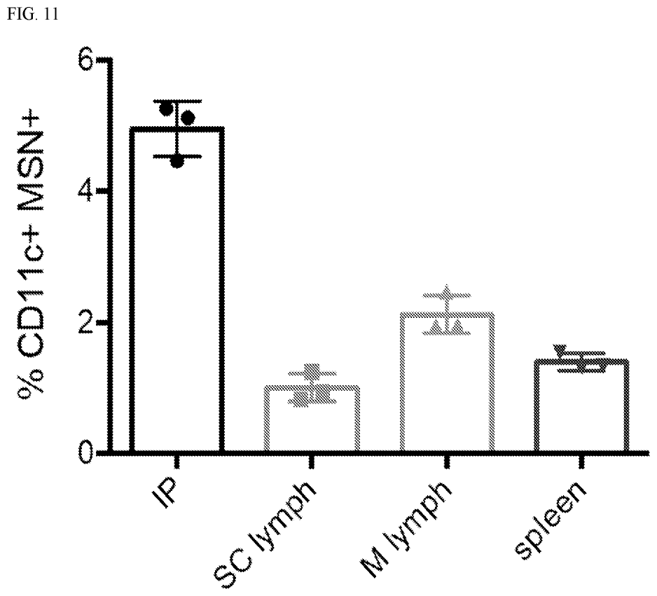

FIG. 11 shows that 5% of cells within the peritoneal fluid of mice 24 hours following intraperitoneal injection with MSN-PEI-CpG-MPL are CD11c⁺ dendritic cells associated with fluorescent MSN-PEI-CpG-MPL. Dendritic cells associated with MSN-PEI-CpG-MPL also account for 1-2% of all cells within lymphatic tissues (i.e., lymph nodes and spleen). Thus, dendritic cells in vivo play an active role in engulfing TLR ligand-bound MSN.

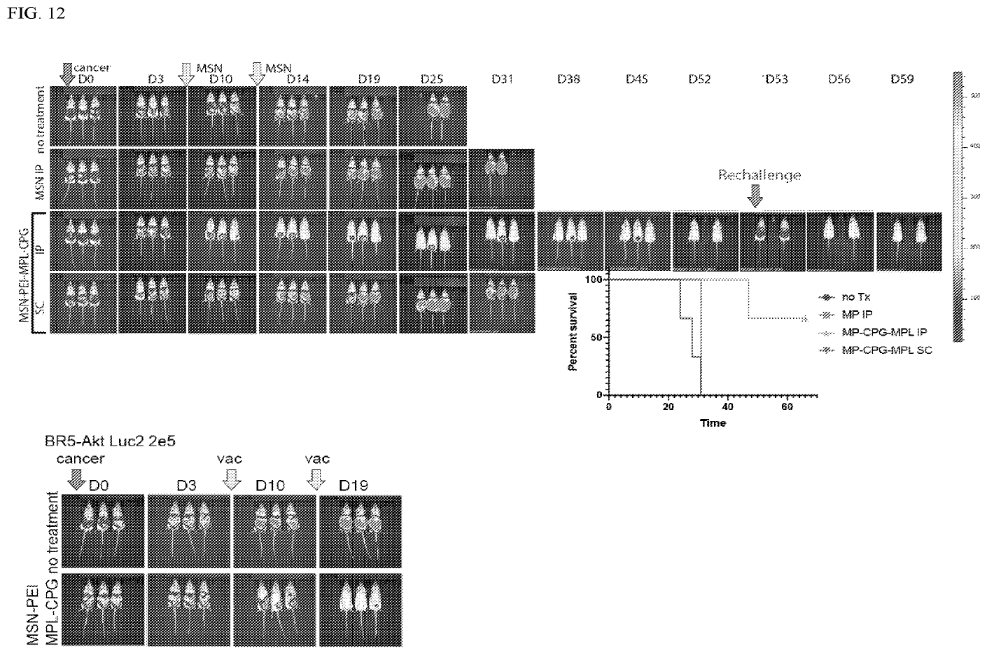

The ability to both activate immune cells and directly kill cancer cells with immunogenic TLR ligand-coated nanoparticles creates an in situ vaccine effect as killed cancer cells release endogenous tumor antigens and immunogenic molecules (e.g., ATP, HMGB1). FIG. 12 shows that mesoporous silica nanoparticles have the ability to eliminate established tumors in mice with ovarian cancer. When “cured” mice were re-challenged with cancer cells, the challenge cancer cells were rapidly eliminated. These result support the existence of memory T cells specific for the cancer cells.

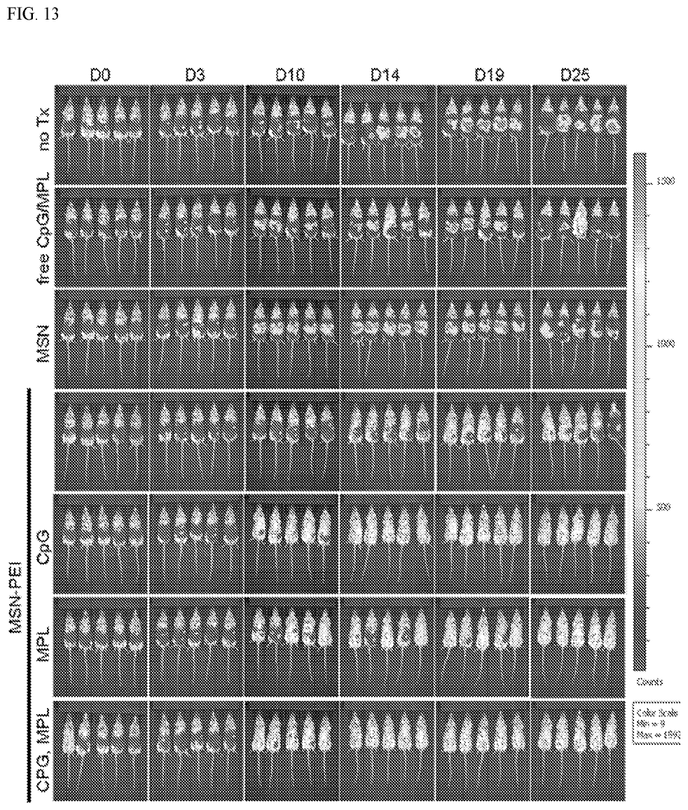

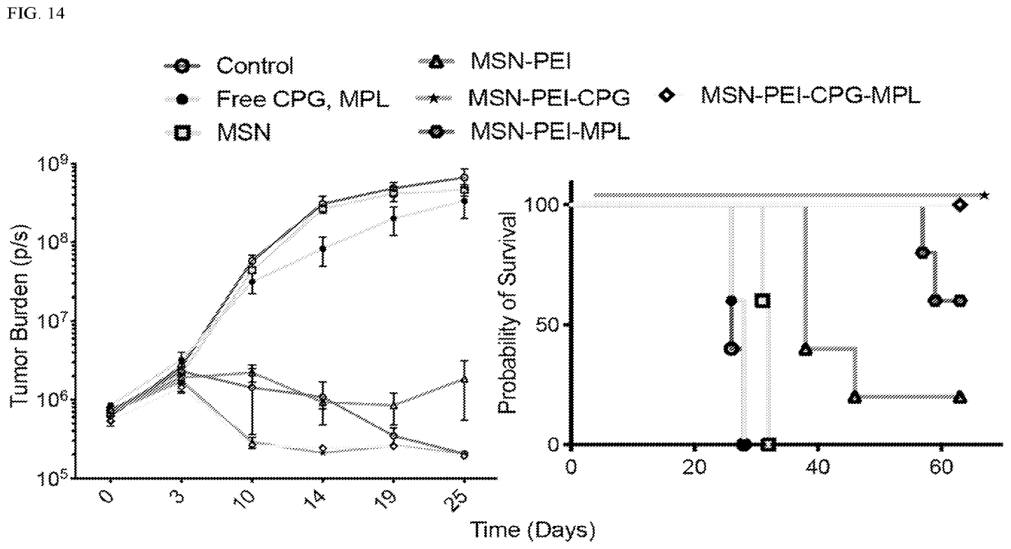

FIGS. 13-14 show that intraperitoneal administration of either free TLR ligand or MSN are ineffective at reducing cancer growth in FVB mice with pre-existing BR5-Akt tumors. Adding PEI and MPL to mesoporous silica nanoparticles increases survival to 60%, while adding CpG, with or without MPL, increases survival to 100%. Thus, the presence of TLR ligands on mesoporous silica nanoparticles promotes eliminating established tumors in the mouse model.

Thus, in one aspect, this disclosure describes a therapeutic platform that includes mesoporous silica nanoparticles whose surface is modified to display an immunomodulatory moiety. As used herein, “mesoporous silica nanoparticles” refers to nanoparticles prepared from mesoporous silica. The nanoparticles may be provided in various shapes, including spherical, oval, hexagonal, dendritic, cylindrical, rod-shaped, disc-like, tubular, or polyhedral.

In some embodiments, a population of mesoporous silica nanoparticles (MSNs) may be monodispersed. Monodispersity can be described as having a polydispersity index (PdI or DPI) of about 0.1 to about 0.2, less than about 0.2, or less than about 0.1.

In another aspect, this disclosure describes a pharmaceutical composition that includes mesoporous silica nanoparticles having surface modifications to display one or more immunomodulatory moieties on the surface of the of MSNs or on the surface and within the porous matrix. The population of mesoporous silica nanoparticles in the composition can be homogeneous or heterogeneous. In a homogeneous population of MSNs, all of the MSNs in the population display the same immunomodulatory moiety. In a heterogeneous population of MSNs, a first subpopulation of MSNs displays a first immunomodulatory moiety and a second subpopulation of MSNs displays a second immunomodulatory moiety. There can be any desired number of subpopulations of MSNs in a heterogeneous composition. Each subpopulation may be surface-modified to display an immunomodulatory moiety independently of any other subpopulation of MSNs in the composition. For example, it is possible to administer a first dose that includes MSNs having a bioactive agent that alters the tumor microenvironment, thereby priming the tumor microenvironment. A second dose can include MSNs having a bioactive agent that kills the cancer cells (e.g., by releasing antigens into the primed microenvironment. When multiple doses are intended, the multiple doses may be administered simultaneously (e.g., a heterogenous population of MSNs) or sequentially, as desired.

The composition may be formulated with a pharmaceutically acceptable carrier to form a pharmaceutic composition. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with a mesoporous silica nanoparticle without causing any undesirable biological effects or interacting in a deleterious manner with any of the other components of the pharmaceutical composition in which it is contained.

The pharmaceutical composition may be formulated in a variety of forms adapted to a preferred route of administration. Thus, a composition can be administered via known routes including, for example, intratumoral, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical can be administered via a sustained or delayed release.

Thus, mesoporous silica nanoparticles may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, solution and the like. The formulation may further include one or more additives including such as, for example, an adjuvant. Exemplary adjuvants include, for example, pathogen-associated molecular patterns (PAMPs), such as Toll-like receptor (TLR) ligands, damage-associated molecular patterns (DAMPs), cytokines, proteins, carbohydrates, lectins, Freund's adjuvant, aluminum hydroxide, or aluminum phosphate.

A formulation may be conveniently presented in unit dosage form and may be prepared by methods well known in the art of pharmacy. Methods of preparing a composition with a pharmaceutically acceptable carrier include the step of bringing mesoporous silica nanoparticles into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of mesoporous silica nanoparticles administered can vary depending on various factors including, but not limited to, the mechanism of action, the specific target, the specific MSNs being administered, the weight, physical condition, and/or age of the subject, and/or the route of administration. Thus, the absolute amount of mesoporous silica nanoparticles included in a given unit dosage form can vary widely, and depends upon factors such as the species, age, weight and physical condition of the subject, and/or the method of administration. Accordingly, it is not practical to set forth generally the amount that constitutes an amount of mesoporous silica nanoparticles effective for all possible applications. Those of ordinary skill in the art, however, can readily determine the appropriate amount with due consideration of such factors.

In some embodiments, the method can include administering sufficient mesoporous silica nanoparticles to provide a minimum dose of, for example, at least 10 μg MSN/kg such as, for example, at least 20 μg MSN/kg, at least 50 μg MSN/kg, at least 100 μg MSN/kg, at least 250 μg MSN/kg, at least 500 μg MSN/kg, at least 1 mg MSN/kg, at least 2 mg MSN/kg, at least 3 mg MSN/kg, at least 4 mg MSN/kg, at least 5 mg MSN/kg, at least 6 mg MSN/kg, at least 7 mg MSN/kg, at least 8 mg MSN/kg, at least 9 mg MSN/kg, at least 10 mg MSN/kg, at least 15 mg MSN/kg, at least 20 mg MSN/kg, or at least 25 mg MSN/kg.

In some embodiments, the method can include administering sufficient mesoporous silica nanoparticles to provide a maximum dose of, for example, no more than 100 mg MSN/kg such as, for example, no more than 50 mg MSN/kg, no more than 40 mg MSN/kg, no more than 30 mg MSN/kg, no more than 25 mg MSN/kg, no more than 20 mg MSN/kg, no more than 15 mg MSN/kg, no more than 10 mg MSN/kg, no more than 5 mg MSN/kg, no more than 2 mg MSN/kg, no more than 1 mg MSN/kg, no more than 500 μg MSN/kg, no more than 250 μg MSN/kg, no more than 200 μg MSN/kg, or no more than 100 μg MSN/kg. MSNs are said to be present in amounts “no more than” a reference amount when MSNs are not absent but are present in an amount up to the reference amount.

In some embodiments, the method can include administering sufficient mesoporous silica nanoparticles to provide a dose characterized by a range having endpoints defined by any a minimum dose identified above and any maximum dose identified above that is greater than the selected minimum dose. For example, in some embodiments, the method can include administering from about 10 μg MSN/kg to about 100 mg MSN/kg to the subject, although in some embodiments the methods may be performed by administering the mesoporous silica nanoparticles in a dose outside this range. In some of these embodiments, the method includes administering sufficient mesoporous silica nanoparticles to provide a dose of from about 10 μg MSN/kg to 50 mg MSN/kg to the subject, for example, a dose of from about 50 μg MSN/kg to 10 mg/MSN/kg, or 1 mg MSN/kg to about 10 mg MSN/kg. In certain embodiments, the method includes administering sufficient mesoporous silica nanoparticles to provide a dose of from 8 mg MSN/kg to 10 mg MSN/kg such as, for example, from 8 mg MSN/kg to 9 mg MSN/kg.

In certain embodiments, the method can include administering an amount of mesoporous silica nanoparticles that is equal to any minimum dose or any maximum dose listed above. Thus, for example, the method can include administering 10 μg MSN/kg, 50 μg MSN/kg, 500 μg MSN/kg, 1 mg MSN/kg, 2 mg MSN/kg, 3 mg MSN/kg, 5 mg MSN/kg, 10 mg MSN/kg, 25 mg MSN/kg, 40 mg MSN/kg, 50 mg MSN/kg, or 100 mg MSN/kg.

As single dose may be administered all at once, continuously for a prescribed period of time, or in multiple discrete administrations. When multiple administrations are used to provide a dose within a certain period, the amount of each dose may be the same or different. For example, a dose of 50 mg MSNs/kg/week may be administered as a single weekly dose of 50 mg MSN/kg, two administrations of 25 mg MSNs/kg on different days within the week, or as a first administration of 30 mg MSNs/kg, followed by a second administration of 20 mg MSNs/kg on different days within the week. Also, when multiple doses are used within a certain period, the interval between doses may be the same or be different.

The mesoporous silica nanoparticles described herein can be used to treat a subject suffering from cancer. Treatment using the methods described herein may result in decreasing the severity of symptoms and/or clinical signs of the condition compared to a similarly situated subject to whom the composition is not administered, and/or completely resolving the condition. Thus, the method includes administering an effective amount of an MSN composition to a subject suffering from cancer. In this aspect, an “effective amount” is an amount effective to reduce, limit progression, ameliorate, or resolve, to any extent, a symptom or clinical sign related to the condition.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Materials

Sodium hydroxide (NaOH), tetraethyl orthosilicate (TEOS), hexadecyltrimethylammonium bromide (CTAB), ammonium nitrate (NH₄NO₃), methanol (MeOH), ethanol (EtOH), LPS-FITC and monophosphoryl lipid A (MPL) were purchased from Sigma-Aldrich (St. Louis, Mo.). CPG ODN was purchased from InvivoGen (San Diego, Calif.) and linear PEI (MW 25,000) was purchased from Polysciences, Inc. (Warrington, Pa.).

### Mesoporous Silica Nanoparticle (MSN) Synthesis

A mixture of water (100 mL), ethanol (40 mL), sodium hydroxide (NaOH, 2 M, 0.75 mL) and cetyltrimethylammonium bromide (CTAB, 0.640 g) was heated to 70° C. under vigorous stirring (750 rpm) in a round bottom flask immersed in an oil bath. Afterwards, tetraethyl orthosilicate (TEOS, 1 mL) was added dropwise to the solution. The TEOS was allowed to undergo a series of hydrolysis condensation reactions for two hours to yield silica CTAB-templated silica nanoparticles. The particles were then isolated by centrifugation (2000×g, 20 minutes) and then washed with MeOH three times. The surfactant removal was performed by suspending the nanoparticles in a solution of 0.45 g/L ammonium nitrate in ethanol and stirring at 60° C. for 20 minutes. Finally, the template-free MSN were consecutively washed twice with water and ethanol and stored suspended in ethanol.

### Preparation of Polyethylenimine (PEI)-Coated Mesoporous Silica Nanoparticles (MSN-PEI)

MSN (0.5 mg) were rinsed twice with water, and then suspended in 1 mL of 0.2 mg/mL PEI in 1X PBS solution. After 10 minutes of rotation at room temperature to allow PEI binding on the MSN surface, the MSN with PEI coating (MSN-PEI) were then rinsed with 1X PBS twice, and stored in 1X PBS.

### Pathogen-Associated Molecular Patterns (PAMPs) Loading

Lipopolysaccharides (LPS) loading. 0.5 mg MSN-PEI were rinsed with 1X PBS, and then suspended in 25 μL of 1 mg/mL LPS in 1X PBS solution. After a 10-minute incubation at room temperature, the LPS-loaded MSN-PEI (MSN-PEI-LPS) were centrifuged at 20,000 relative centrifugal force (rcf) for five minutes to remove extra free LPS and resuspend and stored in 0.5 mL 1X PBS.

Monophosphoryl lipid A (MPL) loading. 0.5 mg MSN-PEI were rinsed with 1X PBS, and then suspended in 25 μL of 1 mg/mL MPL in DMSO solution. After a 10-minute incubation at room temperature, the MPL-loaded MSN-PEI (MSN-PEI-MPL) were centrifuged at 20,000 rcf for five minutes to remove the extra free MPL, then resuspended and stored in 0.5 mL 1X PBS.

CpG loading. 0.5 mg MSN-PEI were rinsed with 1x PBS, and then suspended in 20 μL of 2 mg/mL CpG in double distilled water solution. After a 10-minute incubation at room temperature, the CpG-loaded MSN-PEI (MSN-PEI-CpG) were centrifuged at 20,000 rcf for five minutes to remove extra free CpG, then resuspended and stored in 0.5 mL 1X PBS.

CpG/MPL loading. 0.5 mg MSN-PEI were rinsed with 1x PBS, and then suspended in 20 μL of 2 mg/mL CpG in double distilled water solution. After a 10-minute incubation at room temperature, 25 μL of 1 mg/mL MPL in DMSO solution were added and incubated another 10 minutes. The CpG/MPL-loaded MSN-PEI (MSN-PEI-CpG/MPL) were centrifuge at 20,000 rcf for five minutes to remove extra free CpG and MPL, then resuspend and stored in 0.5 mL 1x PBS.

### Fluorescent Polyethylenimine (PEI) Synthesis

PEI (5 g, 0.2 mmol) was dissolved in 5 mL ethanol and Cy3-NHS or Cy5-NHS (10 mg/mL in dimethyl formamide (DMF, 150 μL, 2 μmol) were added, then the solution was rotated at 40° C. for four days. The mixture was concentrated using a rotary evaporator (1-10 mbar, 40° C.), then 50 μL DMF was added to dissolve any unreacted dye. The mixture was centrifuged (21,000 rcf, 20 minutes) and the isolated pellet (PEI-dye) was dissolved in ethanol and transferred to the rotary evaporator (1-10 mbar, 40° C.) to remove DMF traces. After one hour, the PEI-Cy3 (or PEI-Cy5) were dissolved in 1X PBS at 0.5 mg/mL.

### Loading Efficiency and Stability

PEI-Cy3, LPS-FITC, and CpG-FITC were used to prepare MSN-PEI, MSN-PEI-LPS, MSN-PEI-CpG and quantify loading efficiency of PEI, LPS, and CpG, respectively. MSN samples were rinsed with 1X PBS for variable times to determine stability of modifications. To quantify loading efficiency and stability of MSN samples, the fluorescence was recorded by a microplate reader (BioTek Instruments, Inc., Winooski, Vt.) with excitation/emission at 470/560 nm for PEI-Cy3, and excitation/emission at 488/528 nm for LPS-FITC and CpG-FITC.

### Zeta Potential Measurements

Zeta potential measurements were made using Malvern Zetasizer Nano-ZS (Westborough, Mass.) equipped with a He—Ne laser (633 nm) and non-invasive backscatter optics (NIBS). The samples for zeta potential measurements were suspended in 5 mM NaCl solution in a zeta potential dip cell using the monomodal analysis tool and respecting Smoluchowski model. All reported values correspond to the average of at least three independent samples.

### Incucyte Analysis

BR5 cells were seeded in 12-well plates at a density of 1×10⁵ cells per well. Tumor burden was imaged in vivo using mice with firefly luciferase expressing tumors. Mice were injected with 150 mg luciferin/mg. Mice were anesthetized using 2% isoflurane, and 2D bioluminescence images were acquired using an animal imager (XENOGEN IVIS Spectrum, PerkinElmer, Inc., Waltham, Mass.). ROI measurements of total flux (photons/sec) were acquired using Living Image Software (PerkinElmer, Inc., Waltham, Mass.).

### Cell Lines

The BRCA1-deficient BR5-Akt cell line was generated on an FVB background, while the ID8ova cell line was generated from a spontaneous tumor in C57BL/6 mice. ID8 cells were transformed to express ovalbumin constitutively. Both ID8ova and BR5-Akt models are syngeneic models of high-grade serous epithelial ovarian cancer. To monitor tumor burden using a bioluminescent tag, the cell lines were lentivirus transduced to constitutively express firefly2 luciferase (Luc2). Cell lines were cultured in DMEM containing 10% FBS and 100 units/100 μg penicillin/streptomycin at 37° C. and 5% CO₂. Trypsin-EDTA was used to harvest cells.

### Cell Viability Assay

Cell culture was performed using standard procedures. BR5-AKT cells were maintained in the DMEM containing 10% FBS at 37° C. and 5% CO₂. Cells were passaged at approximately 70% confluency. For cell viability assays, 100 μL of cell suspension (100,000 cells/mL) were seeded into a 96-well plate and cultured for 24 hours at 37° C. The cells were then incubated with 100 μL of different concentrations of silica particles or silicified cells. After a 24-hour incubation, 100 μL of CELLTITER-GLO 2.0 reagent (Promega Corp., Madison, Wis.) was added into each well and incubated for 10 minutes at room temperature. The luminescence readings were then obtained/recorded using a microplate reader (BioTek Instruments, Inc., Winooski, Vt.). The percent cell viability was calculated relative to the control non-treated cells.

### Mouse Models of Ovarian Cancer

Mice were purchased from Charles River laboratories, Inc., Wilmington, Mass.) or The Jackson Laboratory (Bar Harboe, Me.) and housed in a specific pathogen-free facility. All animal protocols were approved by the Institutional Animal Care and Use Committee (IACUC) at the University of New Mexico (Albuquerque, N. Mex.). To generate consistent engraftment and predictable disease progression, 2×10⁵BR5-Akt-Luc2 cells in 200 μL PBS were administered by intraperitoneal (IP) injection in 6-8 week old FVB female mice. Mice were sacrificed when moribund or when weight reached 30 g due to ascites accumulation.

Treatment of Mice with MSN

Tumor-bearing FVB mice were injected intraperitoneally (IP) or subcutaneously (SC) with approximately 200 μg MSN (with TLR ligands as indicated) using doses of 0.174 mg/mouse in 200 μl of PBS at the Day 4 and Day 11. Mice that cleared all tumor cells based on IVIS Spectrum bioluminescent imaging (PerkinElmer, Inc., Waltham, Mass.) were re-challenged with 2×10⁵BR5-Akt-Luc2 cancer cells at a later date, as indicated for each study. All control (no Tx) mice received sham PBS injections (200 μl/mouse).

### Imaging Tumor Burden

For in vivo monitoring of tumor burden, mice with BR5-Akt-Luc2 tumors were administered 150 mg luciferin/kg by intraperitoneal injection, with a 10-minute delay before imaging. Mice were then anesthetized using 2.5% isoflurane, and 2D/3D bioluminescence images were acquired using an animal imager (XENOGEN IVIS Spectrum PerkinElmer, Inc., Waltham, Mass.). ROI measurements of total flux (photons/sec) were acquired using Living Image Software (Perkin Elmer, Inc., Waltham, Mass.).

### Biodistribution of MSN

To study in vivo tissue biodistribution, 200 μg DyLight 633-labeled MSN-PEI-CpG-MPL in 200μL PBS were IP administered to FVB mice 4 days post IP tumor challenge. 24h later, mice were euthanized, and peritoneal tissues were collected and the IVIS Spectrum was used to measure fluorescent intensities.

### In Vivo MSN and DC Interaction

DyLight 633 (Pierce Biotechnology, Inc., Rockford, Ill.)-labeled MSN-PEI-CpG-MPL in 200 μL PBS were administered intraperitoneally (IP) to FVB mice four days post intraperitoneal BR5-Akt tumor challenge. 24 hours later, mice were euthanized, and peritoneal fluid, gut lymph node, subcutaneous lymph nodes (sc lymph), and spleen were collected. Lymph nodes and spleens were mechanically dissociated, and red blood cells were eliminated using a lysing solution (BD PHARM LYSE, BD Biosciences, San Jose, Calif.). Cell suspensions were surface labeled with anti-CD11c FITC (1:250 dilution) at room temperature for 30 minutes in the dark, and then analyzed by flow cytometry (ATTUNE NxT, Thermo Fisher Scientific, Inc., Waltham, Mass.) for double-positive cell populations.

The complete disclosure of all patents, patent applications, and publications, and electronically available material cited herein are incorporated by reference in their entirety. In the event that any inconsistency exists between the disclosure of the present application and the disclosure of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A mesoporous silica nanoparticle comprising:
a mesoporous silica nanoparticle comprising:
a plurality of pores;
a bioactive agent disposed in at least a portion of the pores; and
an outer surface; and
an immunomodulatory moiety, the immunomodulatory moiety either:
disposed in a portion of the pores; or
bound to at least a portion of the outer surface.

2. The mesoporous silica nanoparticle of claim 1, wherein the mesoporous silica nanoparticle comprises a cationic layer disposed on at least a part of the outer surface and the immunomodulatory moiety is bound to at least a portion of the cationic layer.

3. The mesoporous silica nanoparticle of claim 1, wherein the mesoporous silica nanoparticle comprises:
a cationic layer disposed on at least a part of the outer surface; and
an anionic layer disposed on at least a portion of the cationic layer; and the immunomodulatory moiety is bound to at least a portion of the anionic layer.

4. The mesoporous silica nanoparticle of claim 1, wherein the immunomodulatory moiety comprises a pathogen-associated molecular pattern (PAMP), a danger-associated molecular molecule (DAMP), a cytokine, an antibody, or other immunogenic entity.

5. The mesoporous silica nanoparticle of claim 4, wherein the PAMP comprises lipopolysaccharide (LPS), monophosphoryl lipid A (MPL), CpG, R-848, or PolyIC.

6. The mesoporous silica nanoparticle of claim 2, wherein the cationic layer comprises polyethyleneimine (PEI), chitosan, poly(L-lysine), poly(γ-glutamic acid), or a cationic lipid.

7. The mesoporous silica nanoparticle of claim 3, wherein the anionic layer comprises alginate, poly(D-lactic acid), poly(acrylic acid), dextran sulphate, or hyaluronic acid.

8. A pharmaceutical composition comprising the mesoporous silica nanoparticle of claim 1.

9. A method for treating a subject having a tumor, the method comprising administering to the subject a composition that comprises the mesoporous silica nanoparticle of claim 1 in an amount effective to ameliorate at least one symptom or clinical sign of having the tumor.

10. The method of claim 9, wherein the mesoporous silica nanoparticle is administered intratumorally.

11. The method of claim 9, wherein the mesoporous silica nanoparticle is administered intraperitoneally.

12. The method of claim 9, wherein the immunomodulatory moiety comprises a pathogen-associated molecular pattern (PAMP), a danger-associated molecular molecule (DAMP), a cytokine, an antibody, or other immunogenic entity.

13. The method of claim 12, wherein the PAMP comprises lipopolysaccharide (LPS), monophosphoryl lipid A (MPL), CpG, R-848, or PolyIC.

14. The mesoporous silica nanoparticle of claim 3, wherein the cationic layer comprises polyethyleneimine (PEI), chitosan, poly(L-lysine), poly(γ-glutamic acid), or a cationic lipid.
